(19) United States
(12) Patent Application Publication
(10) Pub. No.: US 2017/0122921 A1
(43) Pub. Date: May 4, 2017

(54) Interference Free Gas Measurement

(71) Applicant: Aeroqual Ltd., Auckland (NZ)

(72) Inventors: Geoffrey Stephen Henshaw, Auckland (NZ); Simon James Bennett, Auckland (NZ)

(21) Appl. No.: 15/127,800

(22) PCT Filed: Mar. 26, 2015
(86) PCT No.: PCT/IB2015/001129

§ 371 Date: Sep. 21, 2016

Related U.S. Application Data
(60) Provisional application No. 61/970,564, filed on Mar. 26, 2014.

(51) Int. Cl. G01N 33/00; G01N 27/27; G01N 27/14

(52) U.S. Cl. CPC G01N 33/0059; G01N 33/0039; G01N 33/0032; G01N 27/14; G01N 27/27

(57) ABSTRACT

One or more inexpensive electrochemical gas sensors are paired with a selective ozone sensor. Ozone in ambient air influences the output signals of the electrochemical gas sensors. The unwanted ozone effects are removed from the output signals of the electrochemical gas sensors by comparing them with the selective ozone sensor output signals. The selective ozone sensor signals are removed from and/or added to output signals from the electrochemical gas sensors. True indications of concentrations of the sensed gases in the ambient air result from the compensation for ozone interference.

This application claims the benefit of U.S. Provisional Application No. 61/970,564 filed Mar. 26, 2014, which is hereby incorporated by reference in its entirety as if fully set forth herein.

### BACKGROUND OF THE INVENTION

The cost of traditional monitoring instrumentation for air quality is high, and there is an increasing requirement to lower the cost. One approach is to use less expensive sensors such as electrochemical gas sensors, however such sensors suffer from a lack of selectivity—they respond to gases other than the target gas. It would be advantageous to improve their selectivity.

The measurement of NO₂, SO₂, H₂S and Cl₂ gases in ambient air by electrochemical gas sensors is very difficult, due to the interference by ambient ozone levels. Ozone gas will cause a positive response in NO₂ and Cl₂ electrochemical sensors and a negative response in SO₂ and H₂S electrochemical sensors.

Needs exist for improved air quality sensors.

### SUMMARY OF THE INVENTION

The present invention provides improved air quality sensors at low cost.

It would be advantageous to compensate for the interference by ozone by using a sensor which is selective to ozone, but which is of a similar cost to the electrochemical sensors.

It was discovered that a heated metal oxide sensor operated at high temperature so as to generate a selective response to ozone could be used to compensate for the ozone interference.

If the ozone sensor is co-located with an electrochemical sensor or better still incorporated within the same gas sampling apparatus and data from the sensors is collected at the same time, then the actual NO₂, Cl₂, SO₂ or H₂S concentrations could be calculated using the equation below:

Gas concentration=a*(Electrochemical sensor+/−b*O₃ sensor)+c (Eq1)

where a, b, c are constants which can be calculated through calibration at known humidity, temperature and gas concentrations. The constants may exhibit a dependence on humidity and temperature and therefore it is advantageous to calculate their dependence through calibration and to incorporate temperature and humidity sensors into the gas measurement apparatus to adjust the constants in response to changing gas conditions.

O3 increases a NO sensor response and a Cl₂ sensor response, and the O₃ sensor response must be subtracted in Equation 1. O₃ decreases sensor responses for SO₂ and H₂S, and the O₃ sensor response must be subtracted in Equation 1.

The invention provides an instrument containing a selective ozone sensor and one or more electrochemical gas sensors which exhibit an interfering response to ozone. A microprocessor is connected to the one or more electrochemical gas sensors and to the ozone sensor. An ozone sensor signal from the selective ozone sensor is used to adjust an electrochemical gas sensor output from the one or more electrochemical gas sensors to produce an accurate measurement from the electrochemical gas sensors.

The one or more electrochemical gas sensors are NO₂, SO₂, H₂S and Cl₂ electrochemical gas sensors.

The selective ozone sensor is a heated metal oxide gas sensor.

The electrochemical sensors and the selective ozone sensor are located within 10 meters of each other so that the sensors are sampling substantively the same air parcel at the same time.

The heated metal oxide gas sensor is substantively composed of one or more of WO₃, SnO₂, In₂O₃, MoO₃ or ZnO.

A method of measuring concentrations of one or more of NO₂, SO₂, H₂ S and Cl₂ gases in ambient air uses one or more electrochemical gas sensors. Co-located with the one or more electrochemical gas sensors is a selective ozone sensor. Producing an ozone signal with the selective ozone sensor and using the ozone signal to adjust the one or more signals from the electrochemical gas sensors produces an accurate measurement of the one or more gases.

These and further and other objects and features of the invention are apparent in the disclosure, which includes the above and ongoing written specification, with the claims and the drawings.

### BRIEF DESCRIPTION OF THE DRAWINGS

FIG. 1 is a schematic representation of the new sensor apparatus and method.

FIG. 2 is a graph produced from the new sensor apparatus and method.

### DETAILED DESCRIPTION

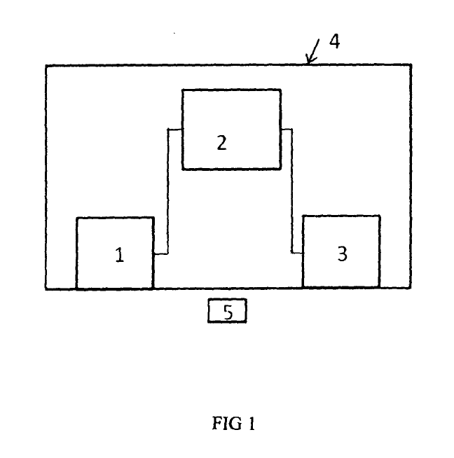

As shown in FIG. 1, a NO₂, SO₂, Cl₂ or H₂S electrochemical sensor 1 has means of contacting gas samples. A microprocessor 2 receives and records sensor outputs, calculates gas concentrations and communicates results to an external logger.

Heated metal oxide ozone sensor 3 has means of contacting the gas sample.

A housing 4 contains the components.

A temperature and relative humidity RH sensor 5 is in contact with a gas sample.

A line power source may be connected to the housing with a step-down transformer, an inverter and resistors for operation the electrochemical gas sensors and the microprocessor and for heating and operating the metal oxide ozone sensor. Operating power may be provided by a battery in the housing or by a low voltage input.

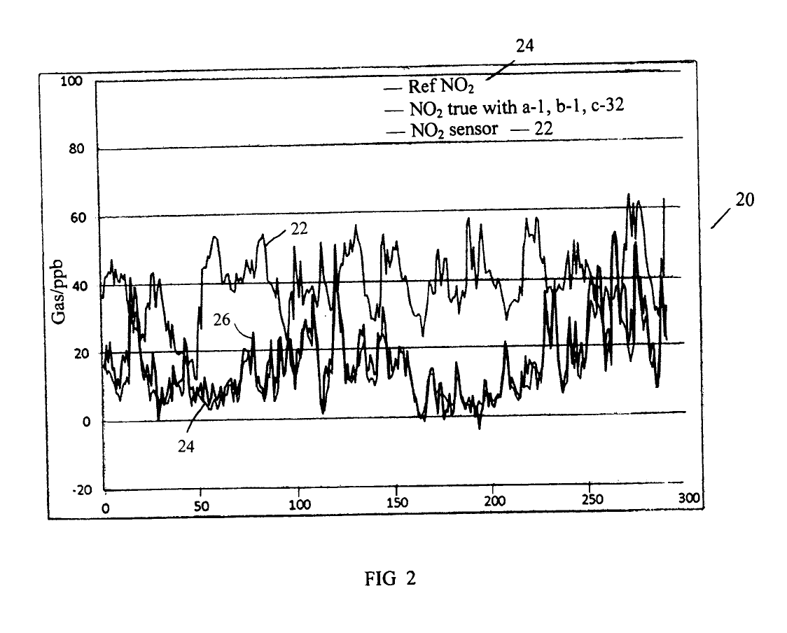

Results of an example are shown in FIG. 2.

The graph shows ambient data 20 from one example using an NO sensor. In this case electrochemical sensor 1 is an NO₂ sensor. The electrochemical NO₂ sensor 1 produces an output signal 22 of parts per billion NO₂. The metal oxide ozone sensor 3 produces an output signal 24 related to parts per billion ozone. Outputs of the No₂ sensor and the ozone sensor are provided to the microprocessor. A reference analyzer using microprocessor 2 subtracts from the NO₂ sensor response 22, the (ref NO₂) response 24. The microprocessor 2 subtracts from the output signal response 22. A part of the ppb is the result of the sensing in NO₂ sensor 1 that O₃ adds to the NO₂ sensor response, and NO₂ true 26 is calculated from the electrochemical NO₂ sensor 1 and a heated metal oxide ozone (O₃) sensor 3 using Eq 1 with a=1, b=1 and c=32 and the +/− sign being a plus. Application of equation 1 has dramatically improved the correlation between the NO2 measured and the reference analyzer. The microprocessor provides an output signal 26 that is the true NO₂ ppb.

NO₂, SO₂, H₂S and Cl₂ sensors 1 are used. The output of the O₃ sensor 3 may be used by subtracting the O₃ sensor output from the NO₂ and Cl₂ sensor outputs and adding the O₃ sensor output to the SO₂ and H₂S sensor outputs. Each electrochemical sensor may have its own associated O₃ sensor, or the output from one O₃ sensor may be stored and used to compensate output from the different electrochemical sensors.

Known temperature and relative humidity effects upon the sensor outputs are used to calculate the true ppb of the sensed gas or gases at standard temperature and relative humidity. For that reason the housing 4 has a temperature and relative humidity sensor 5 attached or close by. An output signal of the temperature and relative humidity sensor 5 may be passed to the microprocessor for compensating the input signals 22 and 24 or their comparison when producing the output signal 26.

The true sensed gas output signal from the housing 4 may be sent to an onboard or remote recorder along with the temperature and relative humidity signal.

While the invention has been described with reference to specific embodiments, modifications and variations of the invention may be constructed without departing from the scope of the invention, which is defined in the following claims.

1. Apparatus comprising an instrument containing one or more electrochemical gas sensors which exhibit an interfering response to ozone, a selective ozone sensor, a microprocessor connected to the one or more electrochemical gas sensors and to the selective ozone sensor, and wherein an ozone sensor output signal from the selective ozone sensor is used by the microprocessor to adjust one or more electrochemical gas sensor output signals from the one or more electrochemical gas sensors to produce accurate gas concentration measurement signal from the one or more electrochemical gas sensors.

2. The apparatus of claim 1, wherein the one or more electrochemical gas sensors (1) comprise NO₂, SO₂, H₂S, NH₃ NO and Cl₂ electrochemical gas sensors.

3. The apparatus of claim 1, wherein the selective ozone sensor comprises a heated metal oxide gas sensor.

4. The apparatus of claim 3, wherein the heated metal oxide gas sensor is substantively composed of one or more of WO₃, SnO₂, In₂O₃, MoO₃ or ZnO.

5. The apparatus of claim 1, wherein the electrochemical sensors and the selective ozone sensor are located within 10 meters of each other, wherein the sensors are sampling substantively the same air parcel at the same time.

6. The apparatus of claim 1, wherein the electrochemical sensors and the selective ozone sensor are located within adjacent housings, wherein the sensors are sampling substantively the same air parcel at the same time.

7. The apparatus of claim 1, wherein the electrochemical sensors and the selective ozone sensor are located within one housing, wherein the sensors are sampling substantively the same air parcel at the same time.

8. A method comprising providing a gas sensing instrument, providing one or more electrochemical gas sensors, measuring concentrations of one or more gases in ambient air using the one or more electrochemical gas sensors, providing a selective ozone sensor, and co-locating the selective ozone sensor with the one or more electrochemical gas sensors, producing an ozone concentration signal with the selective ozone sensor, producing one or more gas concentration signals with the electrochemical gas sensors and using the ozone concentration signal for adjusting the one or more gas concentration signals from the electrochemical gas sensors to produce an accurate concentration measurement of the one or more gases.

9. The method of claim 8, wherein the providing of one or more electrochemical gas sensors (1) comprises providing one or more of NO₂, SO₂, H₂S, NH₃ NO and Cl₂ sensors.

10. The method of claim 9, wherein the measuring concentrations of NO₂, SO₂, H₂S and Cl₂ in ambient air use the instrument of claim 1 wherein each accurate gas concentration equals a*electrochemical sensor reading+/−(b*O₃ sensor reading)+c, wherein a, b, c are determined by calibration of the sensors to O₃ and the sensed gases.
